(19) United States
(12) Patent Application Publication
(10) Pub. No.: US 2008/0015417 A1
(43) Pub. Date: Jan. 17, 2008

(54) Selectively locking minimally traumatic access port

(76) Inventors: David T. Hawkes, Pleasant Grove, UT (US); Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/776,309

(22) Filed: Jul. 11, 2007

Related U.S. Application Data
(60) Provisional application No. 60/830,190, filed on Jul. 11, 2006.

(51) Int. Cl. A61B 1/32

(52) U.S. Cl. 600/219

(57) ABSTRACT

A selectively locking minimally traumatic access port which utilizes a retractor which is configured to create access to a surgical site by displacing surrounding tissues. To minimize the damaged to the displaced tissue, the retractor is further configured to be locked in at least three fixed positions. The medical professional extends and locks the retractor in a position that minimizes the displacement and damage to the surrounding tissues while allowing adequate access to the surgical site.

### BACKGROUND

Traditionally, the surgical exposure employed to perform spinal surgery inflicts significant and long lasting damage to the surrounding soft tissues. Surgical exposure, commonly referred to as an ‘open’ procedure, relies on retraction of muscles to open a channel to the underlying bony structures. Surgical retractors are often used to provide the operating channel. Common surgical retractors as used in the art today include rakes, forks, and different sized and shaped hooks. Normally, the hooks are constructed of a stainless steel or latex-free silicon so that they may be used in the sterile environment of the surgery. While such retractors as rakes or hooks are useful for certain types of surgery, extreme care must be used to ensure that the retractor does not cause additional damage to the wound. In addition, use of the surgical retractor may require two, three, or more additional assistants to the physician, with appropriate training, in order to hold the retractors in the correct position so that the site of the surgery is more easily accessible to the physician. Other types of surgical retractors are inserted into the surgical site and then one or more arms are spread in order to open the insertion site for further access by the physician. These retractors are generally bulky, require substantial training and skill to operate, and user error may increase the difficulty and the time for the surgery. Traditional retraction using the above-mentioned retractors is recognized to cut-off circulation to the muscles and often results in post-operative pain and long-term degradation of muscle function.

Recently, minimally traumatic techniques have been developed to reduce the intra-operative damage and reduce the post-operative recovery time. Using minimally traumatic surgery techniques, a desired site is accessed through portals rather than through a significant incision.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is an isometric view of a selectively locking access port, according to one exemplary embodiment.

FIG. 2 is a partial cut-away side view of a trocar inserted into the patient over a surgical site, according to one exemplary embodiment.

FIG. 3 is a partial cut-away side view of a selectively locking retractor being inserted into a patient, according to one exemplary embodiment.

FIG. 4 is a partial cut-away side view of a selectively locking access port with the retractor locked in a partially extended position, according to one exemplary embodiment.

FIG. 5 is a partial cut-away side view of a selectively locking access port with the retractor locked in a fully extended position, according to one exemplary embodiment.

FIG. 6 is a partial cut-away side view of a selectively locking access port showing mediolateral motion of the cannula portion with respect to the retractor, according to one exemplary embodiment.

FIG. 7A is a perspective view of an assembled minimally traumatic access port configured to be locked in various selective positions, according to one exemplary embodiment.

FIG. 7B is a perspective view of one blade of the minimally traumatic access port including various ratchet slots, according to one exemplary embodiment.

FIGS. 8A-8C illustrate various views of a locking member of the minimally traumatic access port, according to one exemplary embodiment.

FIG. 9A is a side view of the retractor portion of the minimally traumatic access port, according to one exemplary embodiment.

FIG. 9B is sectional view of the retractor portion of the minimally traumatic access port illustrating the engagement of the locking member with the retractor blades according to principles described herein.

FIGS. 9C and 9D are detailed diagrams of the retractor portion of the minimally traumatic access port illustrating the engagement of the locking member with the retractor blades according to principles described herein.

FIGS. 10A-10D illustrate the insertion, engagement, and removal of the selectively locking minimally traumatic access port according to the principles described herein.

FIGS. 11A and 11A are side views showing a two-piece retractor having ratcheting securing mechanisms, according to various exemplary embodiments.

FIG. 12 is a perspective drawing of a four blade retractor and cannula assembly, according to one exemplary embodiment.

FIG. 13 is a perspective drawing of a four blade retractor, according to one exemplary embodiment.

FIGS. 14A-14C illustrate the four blade retractor in varying degrees of extension, according to the principles described herein.

Throughout the drawings, identical reference numbers designate similar, but not necessarily identical, elements.

### DETAILED DESCRIPTION

The present specification describes an apparatus and a method for accessing a surgical site using a selectively locking and minimally traumatic access port. As mentioned above, retractors are used to provide access to a surgery site by displacing and holding tissue away from a surgery site. Using minimally traumatic surgery techniques, portals are inserted through minimal incisions to the surgical site. The medical professional then views and manipulates the surgical site through the portal. The present invention relates to an apparatus and method that uses a selectively locking retractor as part of an access portal. The selectively locking retractor is configured to allow the medical professional to extend and lock the retractor in a plurality of fixed positions. Thus, the medical professional can choose to displace the surrounding tissues to the least amount required to give adequate access to the medical site. By displacing the tissues only as much as is minimally required, the damage to these surrounding tissues is minimized, leading to less patient pain, quicker recovery times, and lower chances of post operation side effects.

According to one exemplary embodiment, the access port consists of two segments: a retractor assembly and a cannula assembly. The retractor assembly consists of two blades that extend to displace tissue surrounding the surgical site to allow access to the desired structure. The retractor is configured to be inserted into the desired position in a stowed or closed configuration, thus minimizing the disturbance of tissues through which it must pass to reach the surgical site. After the retractor has reached the desired position, the retractor blades can be extended to lift the surrounding tissues away from the surgical site. The retractor assembly includes a selectively locking mechanism which allows the medical professional to extend the blades only to the extent necessary to view and manipulate the surgical site.

According to one exemplary embodiment, the cannula assembly attaches to the top of the retractor and creates an operational access port through which the surgical site can be viewed and the necessary procedures performed. Additionally, the cannula provides integrated light, suction, and irrigation capabilities, without interfering with the operational access port. To further facilitate the procedure, the cannula assembly is flexibly attached to the retractor, allowing the cannula assembly to be rotated through various degrees of freedom. This allows a more complete view of the surgical site, better viewing angles, and facilitates the insertion of various surgical implements and hardware.

As used herein, a surgical site includes any subcutaneous location that a medical professional desires to expose by displacing surrounding tissue. A blade includes an element or elements of any geometry that are configured to displace and hold tissue away from a surgical site to allow access or viewing of the surgical site.

While the present apparatus and method may be practiced by or incorporated into any number of systems, the present system and method will be described herein, for ease of explanation only, in the context of a minimally traumatic access portal for use in orthopedic spinal surgery, in which it provides a selectively sized channel to the underlying bony structures of the spine while minimizing trauma to the overlying tissues and tissues that have to be displaced to provide access to the operation site.

In the following description, for purposes of explanation, numerous specific details are set forth in order to provide a thorough understanding of the present systems and methods. It will be apparent, however, to one skilled in the art that the present apparatus, systems and methods may be practiced without these specific details. In other instances, well-known structures associated with the minimally traumatic access port have not been shown or described in detail to avoid unnecessarily obscuring descriptions of the embodiments. Further, a medical professional could perform the described method using steps in an alternative order, insert additional steps, or skip steps according to the medical circumstances and convenience. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

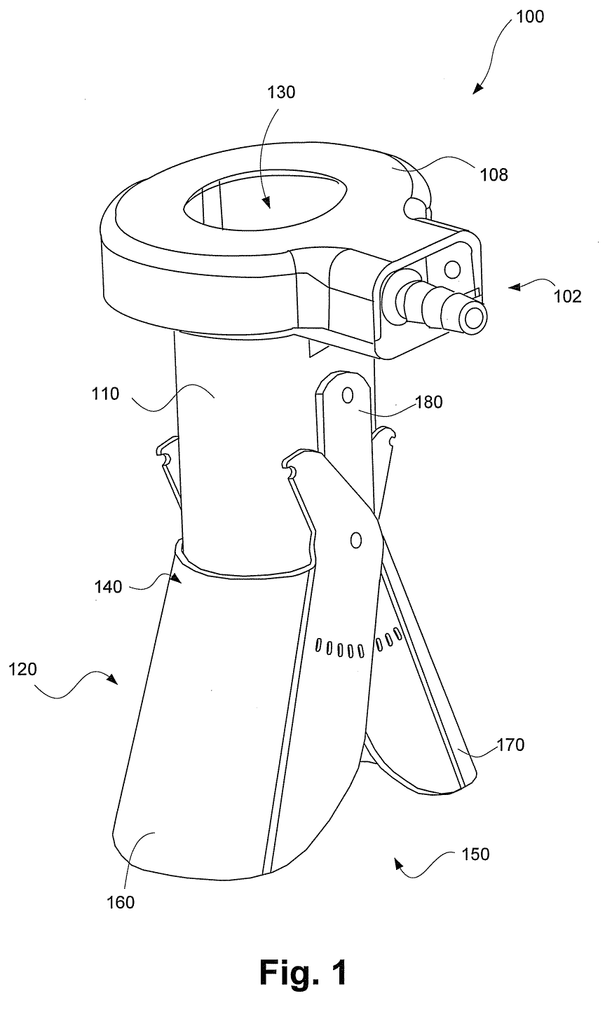

FIG. 1 shows an assembled selectively locking minimally traumatic access port device (100) in a deployed position, according to one exemplary embodiment. As shown, the access port device (100) includes a retractor (120) having a proximal (140) and a distal end (150). The retractor comprises a first retractor blade (160), a second retractor blade (170) and at least one locking member (180). Additionally, a cannula (110) is coupled to the proximal end (140) of the retractor (120). An inner wall of the cannula (110) defines an access port (130). As shown, the exemplary cannula includes a housing (108) on a proximal end of the cannula assembly (110). The housing (108) includes integrated interfaces (102) for fiber optic lights, irrigation, and suction. The access port (130) defined by the body of the cannula assembly (110) is sufficiently large and of an appropriate geometry to allow for the passage of instruments and implants that may be passed through the access port (130) defined by the cannula (110) and into a working space created by the retractor (120). Additionally, the access port (130) may also provide an optical inspection portal, allowing a surgeon to visually inspect the identified surgical location with or without the use of optical cameras and the like.

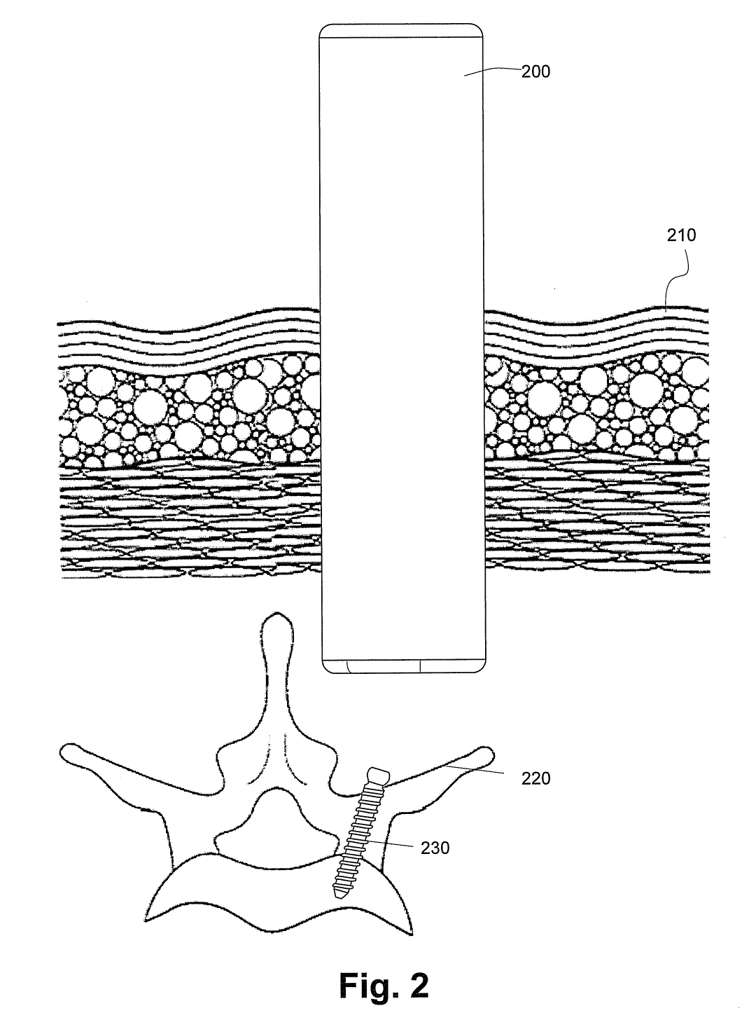

Now referring to FIG. 2, in which the dilation of tissues above the surgical site to allow the subsequent insertion of the less evasive access port (100, FIG. 1) is illustrated. In operation, a k-wire (not shown) may be initially inserted into the soft tissues (210) with the aid of a fluoroscope or by other means. Any number of pedicle screws (230) may then be percutaneously inserted into a desired bone mass, shown in FIG. 2 as a vertebra (220). By way of example, pedicle screw systems may be fixed in the spine in a posterior lumbar fusion then interconnected with rods to manipulate (e.g., correct the curvature, compress or expand, and/or structurally reinforce) at least portions of the spine.

The trocar (200) may then be placed over the k-wire to dilate the soft tissues and provide access to a desired working site. As used herein, the trocar (200) may be any number of stylets used for exploring or dilating tissue. According to one exemplary embodiment, the trocar (200) includes a triangular point on one end. However, the trocar (200) used in connection with the present exemplary minimally traumatic access port device (100) may assume any number of geometric profiles.

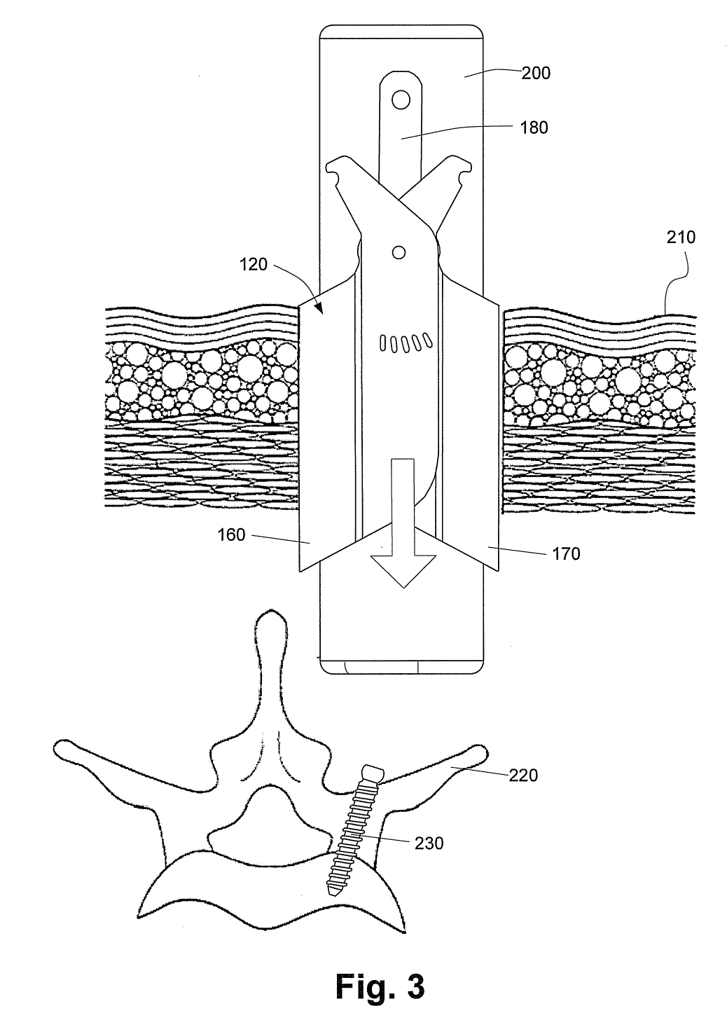

As illustrated in FIG. 3, the trocar (200) has been placed and the retractor (120) is subsequently introduced over the trocar (200) and down to the working site (as illustrated by arrow). As shown in FIG. 2, the retractor (120), in its un-deployed configuration locks the retractor blades (160, 170) adjacent to one another, forming a channel. The trocar (200) can be received within the distal opening of the channel and the retractor (120) may then be slid down the trocar (200) in its un-deployed and minimally invasive state until the distal portion (150, FIG. 1) of the retractor is in a desired working space.

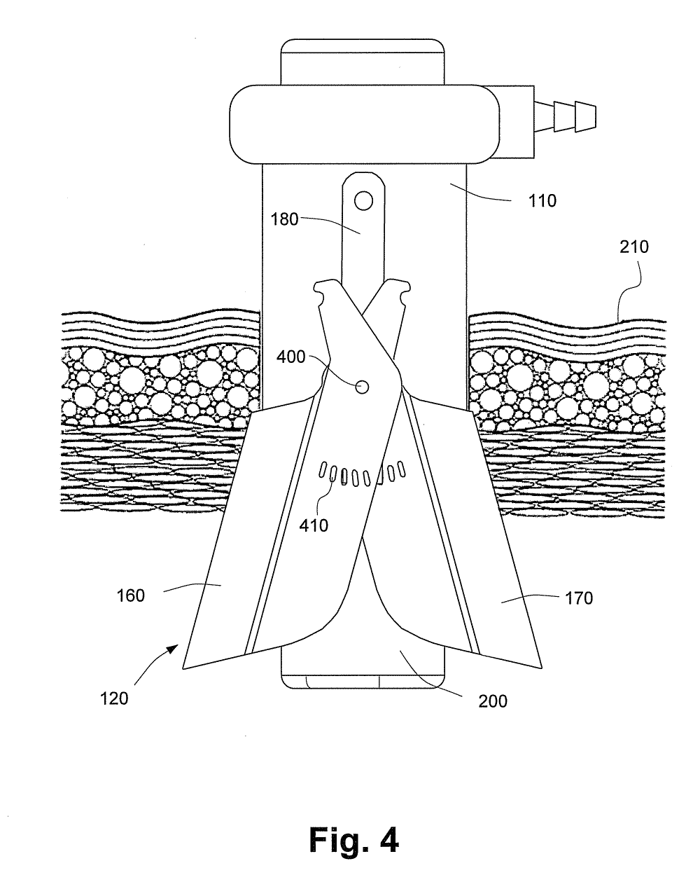

Referring now to FIG. 4, with the retractor (120) appropriately positioned in the desired working space, the cannula assembly (110) is placed over the trocar (200) until it engages the retractor (120). With the minimally traumatic access port device (100, FIG. 1) assembled, the retractor may then be deployed to provide workable access to the vertebra (220, FIG. 3) or other desired structure. The retractor (120) is deployed by opening the two blades (160, 170) which rotate around a pivot fastener (400). The distal ends of the retractor blades (160, 170) spread apart, lifting muscle and tissue from the medical site. The desired medical site may be any acceptable medical site, such as a vertebra (220, FIG. 3) or other location to which a surgeon desires to have clear and clean access. According to one exemplary embodiment, the deployment of the retractor and engagement of the cannula assembly with the retractor may be performed in any order. Prior to deploying the retractor, a series of Cobb elevators and other instruments could be used to subperiosteally dissect the muscle off the facet joints and lamina and spinous processes creating a working space for the retractor to be deployed in.

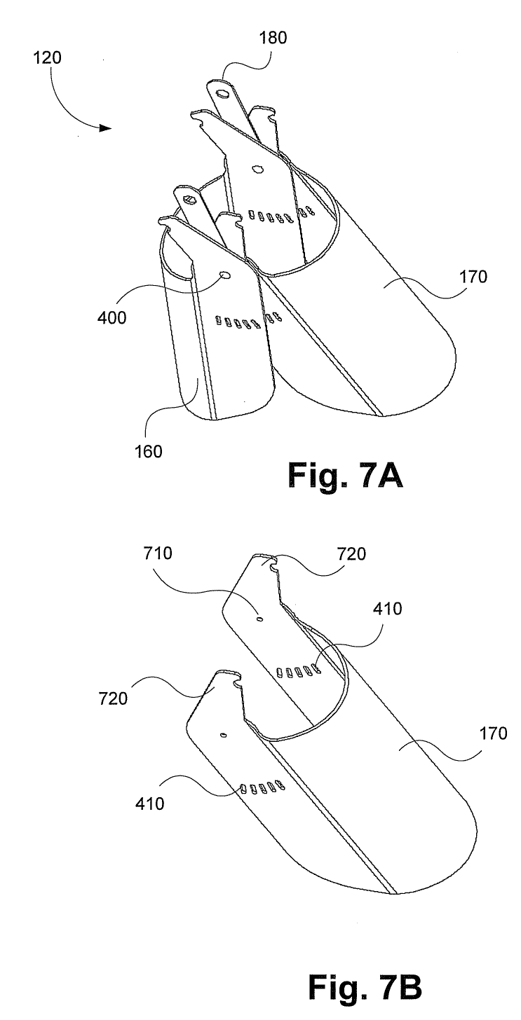

In FIG. 4, the retractor blades (160, 170) are of the present exemplary selectively locking minimally invasive access port (100) are locked in an intermediate position by engaging the locking member (180) with the plurality of ratchet slots (410) defined in the retractor blades (160, 170). The ratchet slots (410) allow the retractor blades to be selectively locked in a plurality of positions, each with a varying amount of angular separation between the retractor blades. The locking mechanism and its method of operation are more fully described in the subsequent FIGS. 7-10. Because the retractor blades can be selectively locked into position, the surgeon is able to access the medical site to the extent necessary to perform the medical task without undue displacement and injury to the tissues surrounding the surgical site.

After the retractor (120) is deployed the trocar (200) can be removed. Once removed, the working space may be accessed for performing decompression, discectomy, interbody fusion, partial facetectomy, neural foraminotomy, facet fusion, posterolateral fusion, spinous process removal, placement of interspinous process distractors, or facet replacement, pedicle replacement, posterior lumbar disc replacement, or any one of a number of other procedures. Irrigation, light and suction functions of the cannula (110) keep the worksite clear of debris and more easily visible.

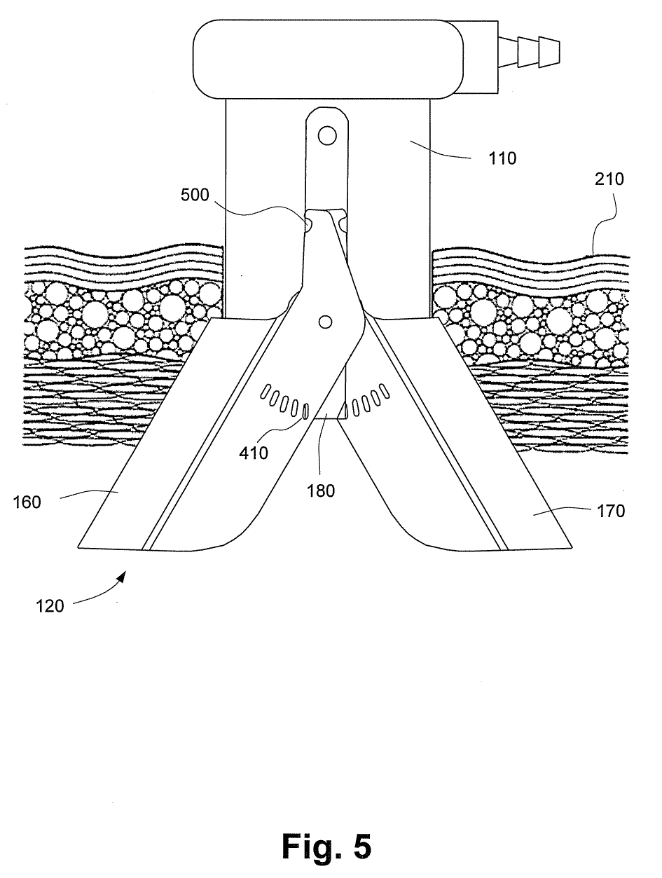

Turning now to FIG. 5, the retractor blades (160, 170) are illustrated in a fully extended position, maximizing the area accessible by the surgeon through the access port, according to one exemplary embodiment. As illustrated in FIG. 5. the locking member (180) is shown to engage a number of innermost ratchet slots (410), locking the blades into position. Semi circular cutouts (500) at the proximal end of the each of the blades provide engagement locations where a tool, such as forceps or specialized pliers, may be used to move the blades to the desired degree of extension.

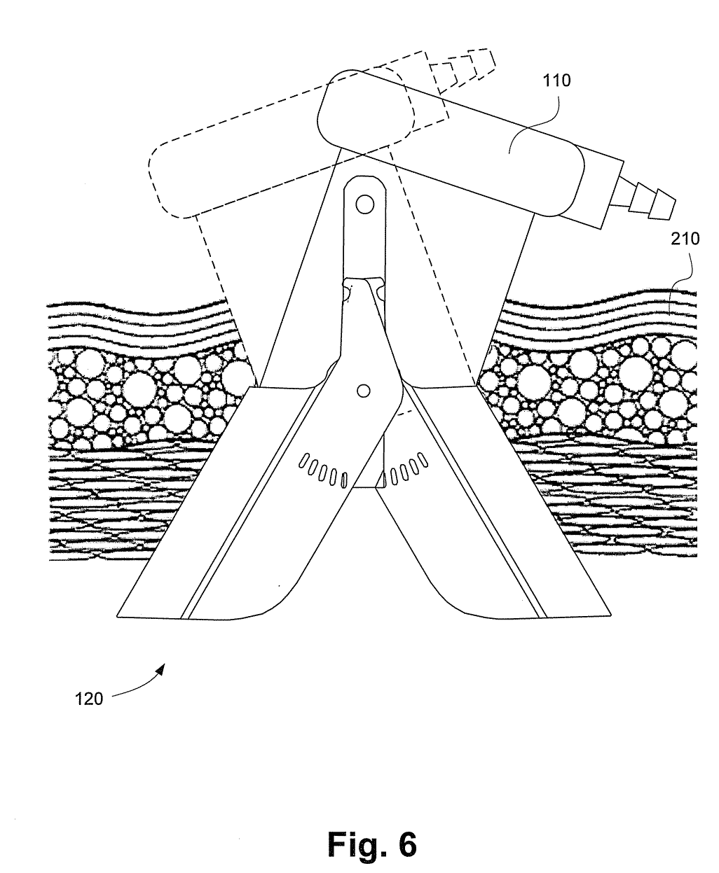

Now referring to FIG. 6, which shows cannula assembly (110) moving in a mediolateral arc within the retractor. According to one embodiment, the motion of the cannula assembly (110) allows visibility and access to the entire working site defined by the retractor (120). Performance of the various procedures via the access port (130, FIG. 1) is facilitated by the rotational freedom provided by the present selectively locking minimally traumatic access port device (100, FIG. 1). Further, the ability to change the orientation of the cannula (110) with respect to the retractor (120) facilitates the insertion of surgical devices and medical hardware.

According to one exemplary embodiment, a variety of methods can be used to flexibly connect the cannula (110) to the retractor (120). By ways of example and not limitation, the cannula assembly (110) could include a two opposing bosses which may engage mating annular groove within the retractor (120) to couple the cannula assembly (110) to the retractor (120), while maintaining the ability to have axial and mediolateral rotation. Another exemplary embodiment includes a hinge interposed between the cannula (110) and the retractor (120) which allows side-to-side movement of the cannula assembly. Further, multiple pivot joints may allow for multidimensional motion of the cannula. According to one exemplary embodiment, a compliant section could be interposed between the cannula (110) and the retractor (120) to allow multidimensional positioning of the cannula (110) through the desired range of motion.

During the medical procedure it may be desirable to hold the cannula (110) at a specific position or location. This can be accomplished through a variety of methods. By way of example and not limitation the cannula assembly can include a specialized attachment point which could be coupled to a positioning arm during an operation. In alternative embodiments, mounts of various size and configuration as are known in the art and could be added to the cannula assembly.

FIGS. 7A through 10D further illustrate the operation of the selectively locking minimally traumatic access port, according to one exemplary embodiment. As illustrated in FIG. 7A, the access port includes a selectively locking retractor (120) including a first retractor blade (160) and a second retractor blade (170) pivotably coupled by a pivot fastener (400). According to one exemplary embodiment, the pivot fastener (400) is used to pivotably couple the first retractor blade (160) and the second retractor blade (170) may include, but is in no way limited to, a rivet, a bolt, or the like. The exemplary selectively locking retractor (120) may also include one or more locking members (180) configured to positionally lock the angular rotation of the retractor blades about the pivot fastener (730), when engaged.

FIG. 7B further illustrates the construction of the retractor blades (160, 170) of the present exemplary embodiment. As shown in FIG. 7B, each retractor blade may include a plurality of proximal arms (720). As shown, a plurality of ratchet slots (410) configured to enable the selectively locking functionality of the exemplary less invasive access port can be formed in an arcuate pattern having a substantially consistent radius from a fastener orifice (710).

Further, while the retractor blades (160, 170) of the exemplary selectively locking retractor (120) have been described and illustrated as having a particular shape, the retractor blades (160, 170) may assume any number of shapes, and may be made of any number of materials to satisfy a desired surgical purpose. Further, the retractor blades need not be substantially identical. Rather, elements with different and distinct geometry could be coupled together to serve as selectively locking retractor blades.

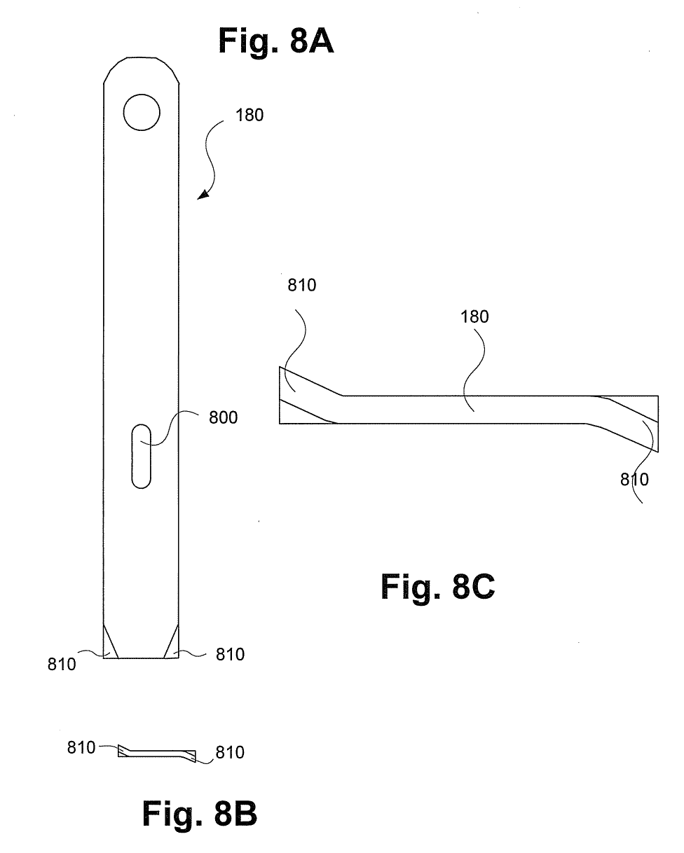

In the exemplary embodiment shown in FIG. 7A, locking members (180) are configured to interact with the arcuate pattern of ratchet slots (410). The geometry of one exemplary embodiment of the locking members (180) is further described in FIG. 8A through 8C.

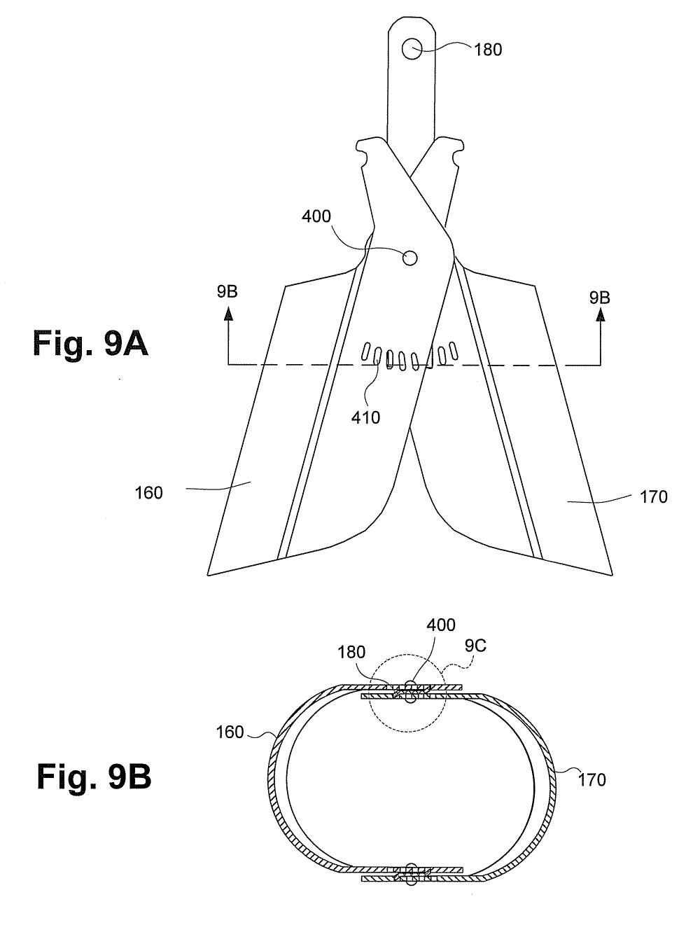

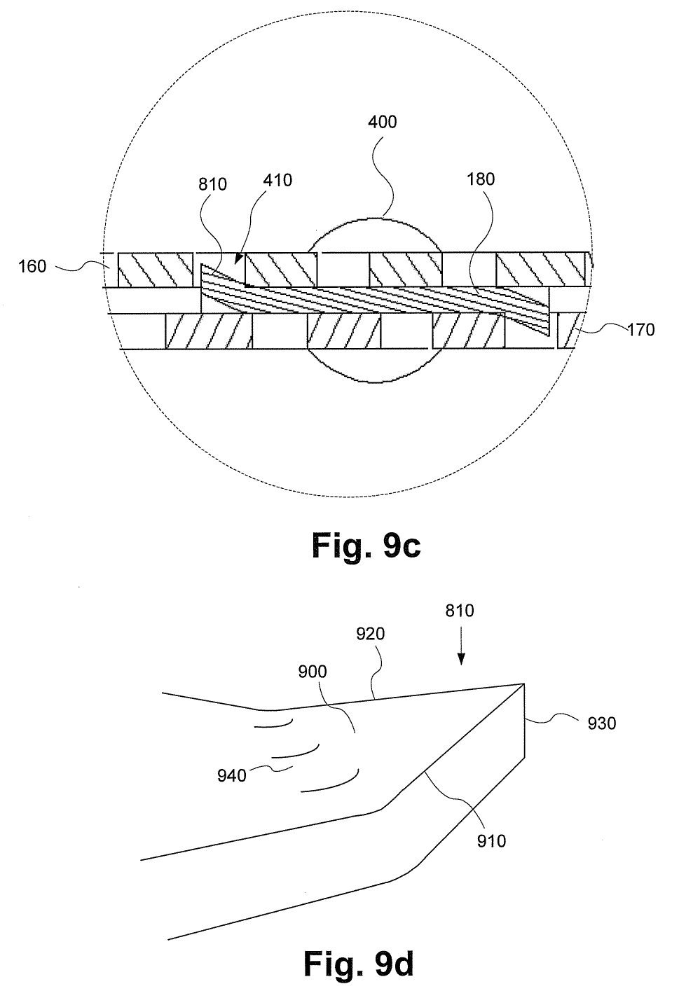

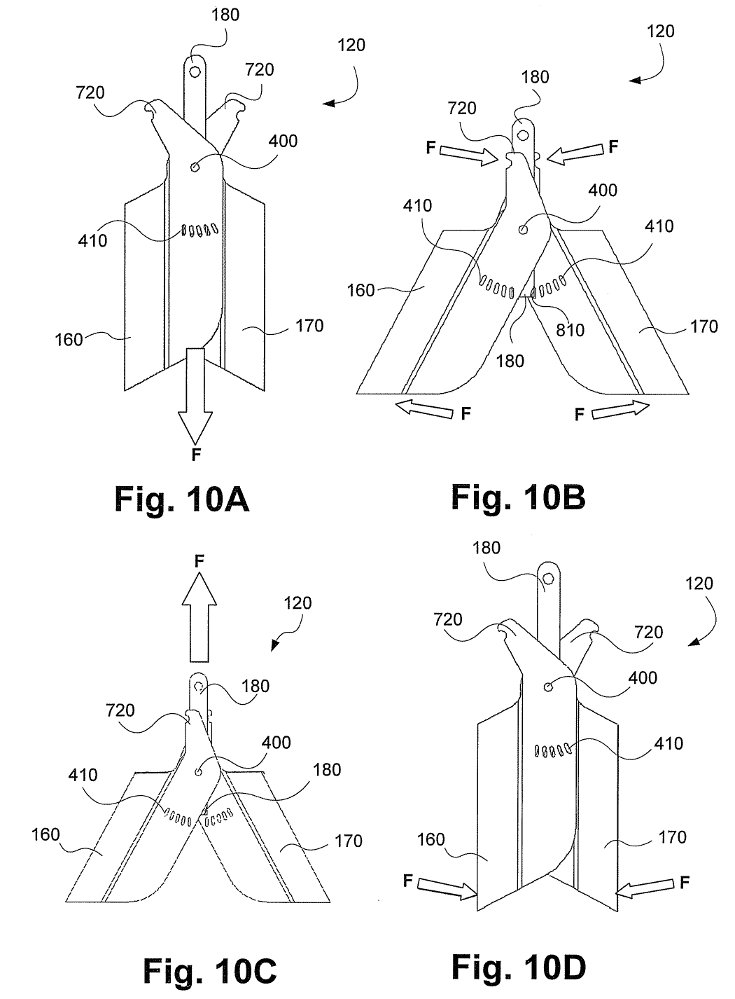

Referring now to FIG. 8A through 8C, one exemplary embodiment of the locking members (180) is illustrated. As shown in FIGS. 8A through 8C, the locking members (180) may include a substantially planar body including an elongated engagement slot (800) configured to mate with the fastener orifice (710, FIG. 7) of the retractor blade (160, 170; FIG. 7) and receive the pivot fasteners (730, FIG. 7). Additionally, as illustrated in FIGS. 8A through 8C, the distal end of the locking member (180) includes a plurality of catch tabs (810) or engagement features configured to selectively interact with the ratchet slots (410, FIG. 4) of the retractor arms. As shown, the catch tabs (810) may, according to one exemplary embodiment, be formed on opposing sides of the locking member (180) and may further be oriented in differing directions, as illustrated in FIGS. 8B and 8C. FIG. 8B shows an end view of the locking member (180) while FIG. 8C is an enlarged drawing of the same end view as shown in FIG. 8B. According to one exemplary embodiment, the catch tabs (810) formed on the distal end of the locking member (180) may be formed by bending the corners of the locking member. Alternatively, any number of other engagement features may be used in place of the catch tabs (810) including, but in no way limited to, a machined protrusion, a fastened protrusion, and the like. Further details of the operation of the retractor blades (160, 170) in combination with the locking members (180) will be provided below with reference to FIGS. 9A through 10D.

FIGS. 9A through 9C illustrate an exemplary interaction between the locking members (180) and the retractor blades (160, 170), when assembled. As shown in FIGS. 9A through 9C, the assembled locking retractor (180) overlaps a plurality of retractor blades (160, 170) with a common union being formed at the pivot fastener (400). Additionally, as shown, the locking members (180) are disposed between the adjacent walls of the first retractor blade (160) and the second retractor blade (170). When installed, the catch tabs (810) formed on the distal end of the locking members (180) may selectively engage the ratchet slots (410).

In one exemplary embodiment shown in FIG. 9C, the catch tabs (810) are formed with first catch tab extending upward and a second catch tab extending downward. The first catch tab extends upward and engages ratchet slots on the first retractor blade (160) and the second catch tab extends downward and engages ratchet slots in the second retractor blade (170).

FIG. 9D shows a detailed view of a single catch tab (810). As previously mentioned, the catch tab (810) may be formed, according to one exemplary embodiment, by bending the corner of the locking member (180). According to one exemplary embodiment, the bend (940) changes the angle of the surface of the locking member (180) forming a first angled edge (910) and a second angled edge (920). The first angled edge (910) creates an incline over which the edges of the ratchet slots (410, FIG. 9D) can pass when the retractor blades (160, 170) are extended. This incline allows the retractor blades (160, 170) to be extended with minimal force. During the extension of the retractor blades, each of the ratchet slots (410) sequentially encounters the corresponding catch tab (810). When a ratchet slot (410) first encounters the catch tab (810), the catch tab springs into the slot cavity. Then, as the retractor blades (160, 170) continue to extend, the edge of the slot climbs up the incline formed by angle (910) until the slot passes over the catch tab. When the desired amount of extension has been achieved, the extending force is removed and the retractor blades (160, 170) move in a retrograde fashion until an inside edge of a ratchet slot (410) engages the outside edge (930) of the catch tab (810). The retractor blades (160, 170) are then locked in position and can only be retracted when the locking member (180) is withdrawn from engagement with the slots.

The locking member (180) is withdrawn from engagement with the slots by exerting an upward force on the locking member sufficient that the pivot fastener (730) is translated in the engagement slot (800) of the locking member (180) such that the radius from the pivot fastener (400) to the end of the catch tabs (810) is less than the radius of the arcuate ratchet slots (410). The incline formed by the second edge (920) allows the top edge of the currently engaged ratchet slot (410) to slide along the incline formed by the second edge (920), thus minimizing the force necessary to disengage the locking member (180) from the ratchet slots (410). The interaction of the locking member with the ratchet slots of the retractor blades is further described in FIGS. 10A through 10D.

Now referring to FIGS. 10A through 10D, which illustrate the operation of the present exemplary selectively locking retractor (120), the expandable retractor (120) is first inserted into an opening in the tissue. When inserted, the distal portions of the retractor arms (160, 170) are placed together in a closed position to minimize the size of opening needed for insertion. When in the closed position, the catch tabs (810) may be engaged in the outermost ratchet slots (410) of the retractor arms (160, 170). Throughout FIG. 10, arrows indicate the direction of force (F) used to effectuate the motion described. The arrow of FIG. 10A represents the downward force required to insert the retractor into the opening in the tissue.

Once inserted into a patient, the present exemplary selectively locking retractor may be opened. As shown in FIG. 10B, an instrument, such as forceps or specialized pliers may be used to bring the proximal arms (720) together, causing the distal portions of the retractor arms (160, 170) to ratchet open on the locking member (180). Specifically, the catch tabs (810) may, if enough force is exerted on the proximal arms, pass through the various ratchet slots (410) until the catch tabs are in a desired slot and the opening force is removed. According to this exemplary embodiment, the retractor arms (160, 170) of the present exemplary locking retractor (1600) may be fixedly positioned in any number of desired open positions, thereby providing a surgeon with the ability to limit trauma to an operating site if a full opening is not necessary or appropriate for a desired procedure. According to one exemplary embodiment, the present exemplary locking retractor may be fixedly locked in three or more positions.

Once the desired procedure is performed, the selectively locking retractor (120) may be closed using the steps illustrated in FIGS. 10C and 10D. As shown in FIG. 10C, the locking members (180) may be pulled up to draw the catch tabs (810) out of the ratchet slots (410) on the retractor arms (160, 170), releasing the locking mechanism. According to the exemplary embodiment illustrated in FIG. 1C, when the locking members (180) are pulled up, the pivot fastener (400) is translated in the engagement slot (800, FIG. 8) of the locking member (180) such that the radius from the pivot fastener (400) to the end of the catch tabs (810) is less than the radius of the arcuate ratchet slots (410). Consequently, the retractor arms (160, 170) may be rotated without interaction between the catch tabs (810) and the ratchet slots (410). With the catch tabs (810) out of contact, the port is then able to close again for removal from the wound, as illustrated in FIG. 10D.

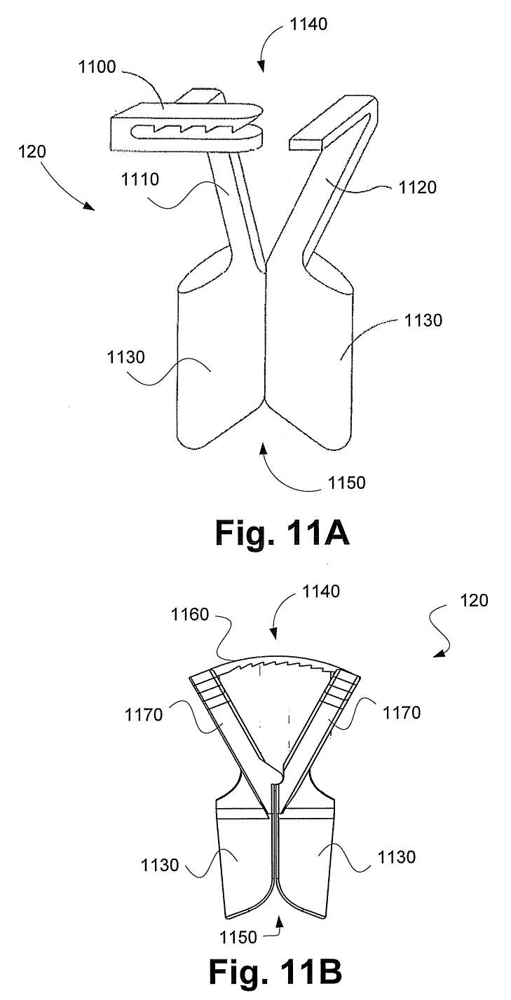

FIG. 11A illustrates a retractor (120) including an alternative securing mechanism (1100), according to one exemplary embodiment. Specifically, FIG. 11A shows a two-piece retractor (120) having a ratcheted retaining mechanism (1100) affixed to at least one arm (1110) of the retractor. According to the exemplary embodiment illustrated in FIG. 11A, a second locking arm (1120) of the retractor (120) may include a protrusion configured to be securely received by the retaining mechanism (1100) when the retractor is in a deployed position with the retractor blades (1130) separated. One advantage of the exemplary two-piece retractor (120) over traditional retractors is that by designing the retractor so that the proximal portion (1140) and the distal portion (1150) operate in opposing directions, the locking mechanism or securing device may be positioned on the proximal portion of the retractor (120). Consequently, in contrast to traditional retractors, the locking mechanism will be located outside of the wound during a medical procedure, providing convenient access to a surgeon for deployment and/or retraction. Additionally, the ability to draw in the retractor blades (1130) after deployment allows the present two-piece retractor (120) to be re-useable.

Similarly, FIG. 11B illustrates yet another alternative securing mechanism (1160), according to an alternative embodiment. As shown, the ratcheting securing mechanism (1160) may be a curved toothed member configured to enable locking of the arms (1110, 1120) at any position. Consequently, the illustrated securing member (1160) can be used to secure the position of the locking arms (1170), and consequently the retractor blades (510) in any number of deployed stages.

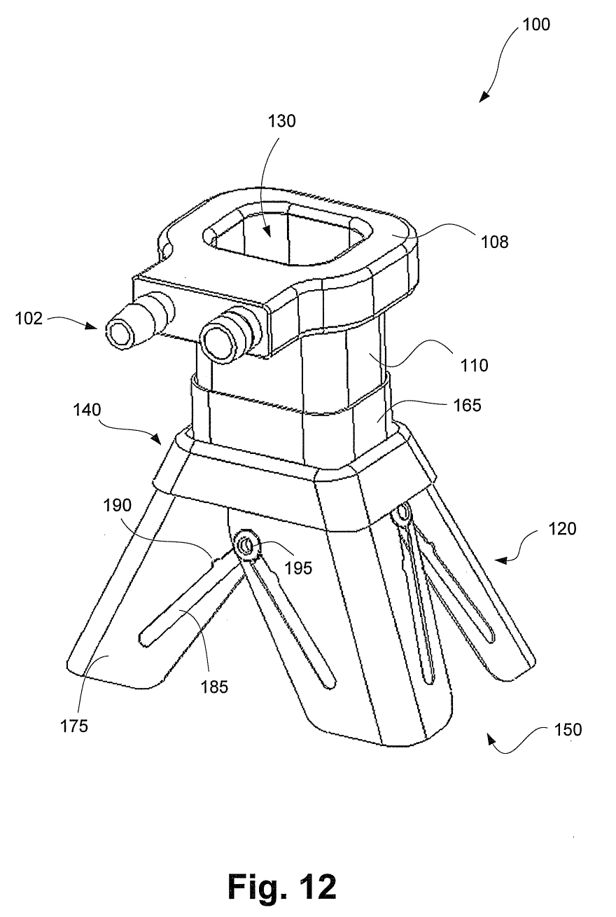

Referring now to FIG. 12, another exemplary embodiment of an assembled selectively locking minimally traumatic access port device (100) is illustrated. As shown in FIG. 12, the access port device (100) includes a four blade retractor (120) having a proximal (140) and a distal end (150). The retractor comprises a plurality of retractor blades (175) that extend to displace tissue from the surgical site. The motion of the retractor blades (175) is constrained by sliding pins (195) that translate within guide tracks (185). Positioned in the edge of the guide track (185) is a detent (190) configured to receive the sliding pin (195). Additionally, a cannula (110) is coupled to the proximal end (140) of the retractor (120). An inner wall of the cannula (110) defines an access port (130). As previously described, the exemplary cannula includes a housing (108) on a proximal end of the cannula assembly (110) and includes integrated interfaces (102) for fiber optic lights, irrigation, and suction. The access port (130) allows the observation and manipulation of the working space created by the retractor (120). A silicone sleeve (165) surrounds the connection between the cannula (110) and the retractor (120).

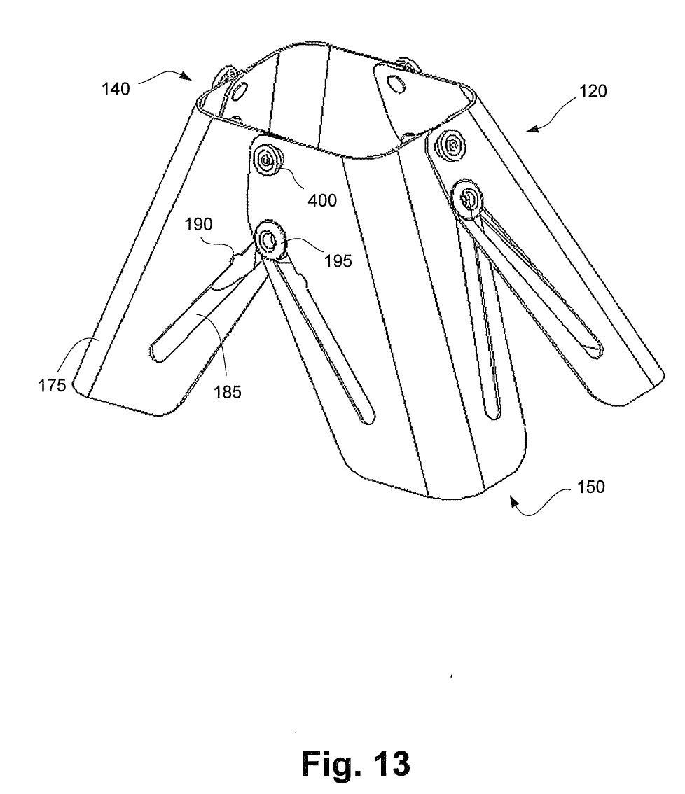

Referring now to FIG. 13, a perspective view of an exemplary embodiment of a four blade retractor (120) is illustrated. Each blade (175) of the exemplary four blade retractor (120) is connected at the proximal end (140) by a pivot fastener (400) to the adjoining blade. Additionally each blade contains two guide tracks (185) configured overlap with guide tracks (185) of adjoining blades (175). A sliding pin (195) passes through the overlapping openings of the guide tracks (185) and controls the motion of the blades as they are extended. Positioned along the guide track is at least one detent (190) configured to receive the sliding pin (195). According to the present exemplary embodiment, when the sliding pin (195) received by a detent (190), an intermediate stop is created such that the retractor blades are partially extended or locked in a partially opened position. Particularly, as illustrated in FIG. 13, the separate guide tracks allow the retractor to be deployed independently in the medial-lateral and superior-inferior directions. The force required to extend or pull in the retractor blades may, by way of example and not limitation, be exerted through the sliding pin (195). When the sliding pin or pins (195) are pushed downward, a corresponding force is generated which tends to extend the retractor blades (175). Conversely, when the sliding pin or pins (195) are pulled upward, the corresponding force is generated which tends to pull in the retractor blades (175).

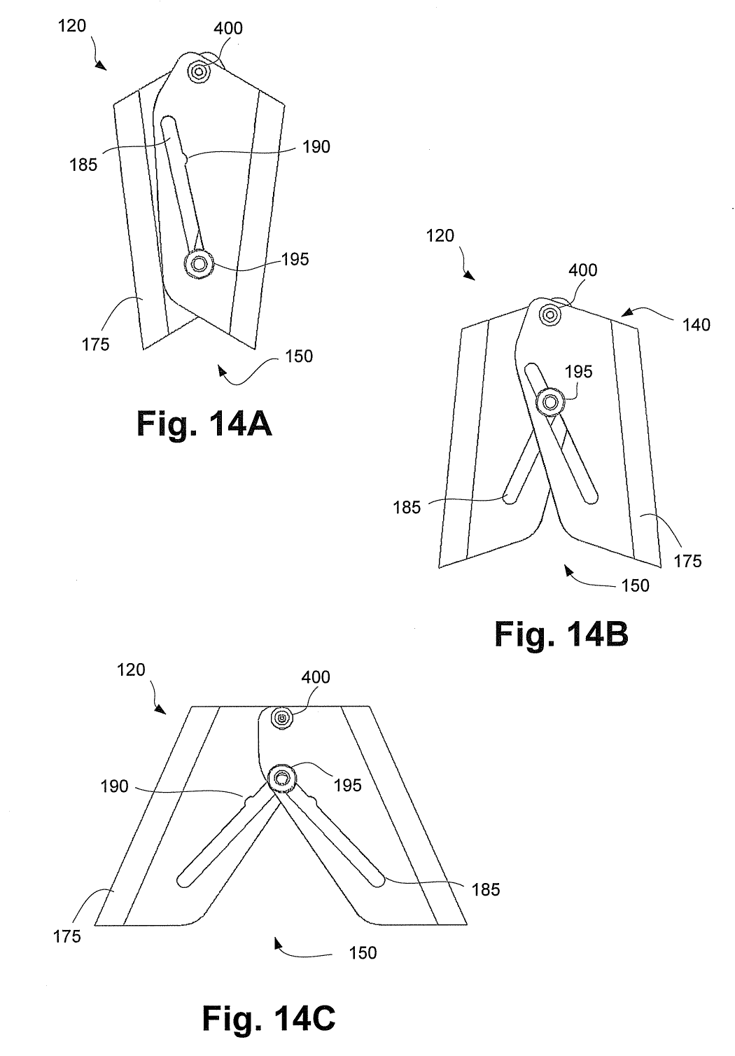

FIGS. 14A though 14C show an exemplary retractor in three configurations: stowed, partially extended, and fully extended, according to one exemplary embodiment. Particularly, FIG. 14A shows the retractor blades in a stowed position for insertion through an opening in the soft tissues to the surgical site. In the stowed position the retractor blades are rotated about pivot fastener such that the sliding pin moves to the bottom of the guide track. The sliding pins (195) prevent the further inward motion of the retractor blades (175) by contacting the distal end of the guide tracks (185).

FIG. 14B shows the retractor blades in a partially extended configuration. When the sliding pin (195) is forced toward the proximal end of the retractor (140), the retractor blades (175) extend. When the sliding pin (195) reaches the at least one detent (190), the sliding pin (195) is received into the at least one detent (190), such that a stable and partial deployed configuration is achieved.

FIG. 14C shows the retractor (120) in the fully extended position. In the fully extended position, the slider pin (195) is translated to the most proximal position in the guide track (185) and prevents the further outward motion of the retractor blades (175).

In conclusion, the selectively locking retractor is configured to be inserted in a stowed configuration through tissue to the surgery site. Once positioned over the surgery site, the medical professional can extend and lock the retractor in a plurality of fixed positions. Thus, the medical professional can choose to displace the surrounding tissues to the least amount required to give adequate access to the medical site. By displacing the tissues only as much as is minimally required, the damage to these surrounding tissues is minimized, leading to less patient pain, quicker recovery times, and lower chances of post operation side effects.

Further, the present exemplary systems and methods allow for a surgeon to manipulate the viewing angle of the minimally traumatic access port into the working site in a transverse plane. Manipulation of a port medially and laterally facilitates: decompression of the neural elements; simple access to the contralateral side of the spine, eliminating the need to place a tube through the skin on that side; access to the transverse process on the ipsalateral side for a posterolateral fusion, and generally simplifies a surgical procedure by increasing the surgeon's viewing of the surgical site. Further, the present exemplary systems and methods allow for the retraction of muscles rather than the distal lifting of muscles during procedures. Additionally, the present exemplary system positions the arm securing mechanism outside of the wound where it may be readily accessed by the surgeon.

Moreover, the present system and method do not require the additional use of a light source, a suction device, and an irrigation device because these items are integral to the construction of the minimally traumatic access port device. Existing access ports require the additional use of a light source, a suction device, and an irrigation device, all of which decrease the space left for surgical instruments and for viewing of the surgical site.

Further advantages of the present exemplary system include the variety of materials, including composites, plastics and radio-opaque materials, that the cannula and retractor can be made from. Existing access ports are made of metal, which has several shortcomings: metal conducts electricity which can cause arcing from an electrocautery device and thus unwanted stimulation of the nerves; metals are reflective and produce an environment that is difficult to clearly view the surgical site; metals are radio-opaque and make intra-operative x-ray difficult. Alternative materials that are partially radio-opaque would provide for optimal intra-operative x-ray. The geometry and structural integrity of the prior art does not allow for the use of alternative materials.

The preceding description has been presented only to illustrate and describe embodiments and examples of the principles described. This description is not intended to be exhaustive or to limit these principles to any precise form disclosed. Many modifications and variations are possible in light of the above teaching.

What is claimed is:

1. An access port, comprising:
a retraction means, said retraction means being configured to displace tissue, said retraction means being configured to lock in at least three fixed positions.

2. The access port of claim 1 wherein said retraction means comprises at least two retractor blades, said at least two retractor blades being configured to lock in at least three fixed positions.

3. The access port of claim 2 further comprising a locking means, said locking means comprising an engaging means and a receiving means, wherein said receiving means and said engaging means are configured to selectively lock said at least two retractor blades in at least three fixed positions.

4. The access port of claim 3, wherein said at least two retractor blades are pivotally coupled.

5. The access port of claim 4, wherein said at least two retractor blades comprise a first retractor blade and a second retractor blade pivotally coupled;
wherein said first retractor blade and said second retractor blade each having a distal and a proximal end, said distal end of said first retractor blade and said distal end of said second retractor blade each being configured to displace body tissue and said proximal end of said first retractor blade configured as said engaging means and the proximal end of said second retractor blade configured as said receiving means.

6. The access port of claim 5, wherein said engaging means comprises a ratchet having a plurality of teeth and wherein said receiving means is configured to capture said plurality of teeth such that said first retractor blade and said second retractor blade can be locked in at least three fixed positions.

7. The access port of claim 5, wherein said receiving means comprises a slot containing a plurality of teeth and said engaging means includes said proximal end of second retractor blade, said receiving means being configured to capture said proximal end of said second retractor blade such that said first retractor blade and said second retractor blade are lockable in at least three fixed positions.

8. The access port of claim 4, further comprising:
a retractor including a first retractor blade and a second retractor blade wherein said first retractor blade and said second retractor blade are pivotally connected by a pivot fastener;
at least one locking member coupled to said pivot fastener.

9. The access port of claim 8, wherein said at least one locking member further comprises:
a generally planar body having distal and a proximal end;
an elongated orifice defined in said generally planar body; and
a plurality of engagement members disposed on said distal end of said generally planar body.

10. The access port of claim 9, further comprising:
a plurality of ratchet slots defined in each of said first and second retractor blades;
wherein said at least one locking member is configured to selectively engage said plurality of ratchet slots to positionally lock said first and second retractor blades.

11. The access port of claim 10, wherein said plurality of engagement members disposed on said distal end of said generally planar body protrude into a frontal plane of said generally planar body and a rear plane of said generally planar body.

12. The access port of claim 11, wherein said plurality of ratchet slots are defined in each of said first and second retractor blades in an arcuate orientation having a consistent radius from said pivot fastener; wherein said plurality of engagement members are disposed a fixed distance from said elongated orifice, said fixed distance being substantially equal to said consistent radius.

13. The access port of claim 12, wherein said at least one locking member is translatable along said pivot fastener to selectively engage and disengage said plurality of engagement members from said plurality of ratchet slots.

14. A method of selectively locking a position of a retractor having plurality of retraction blades comprising selectively inserting a protrusion in an orifice of a first retractor blade and an orifice in a second retractor blade.

15. A minimally traumatic access port, comprising:
a retractor having a plurality of retractor blades, said retractor blades having at least one guide track, said at least one guide track being configured to receive a translating member such that said translating member engages with said at least one guide track to constrain the motion of said retractor blades.

16. The minimally traumatic access port of claim 15, wherein said translating member engages said guide track in at least three locations such that said plurality retractor blades are fixed in at least three corresponding positions.

17. The minimally traumatic access port of claim 16, wherein at least two guide tracks are configured to allow said plurality of retractor blades to be deployed independently in a medial-lateral direction and in a superior-inferior direction.

18. The minimally traumatic access port of claim 17, wherein said at least two guide tracks further comprise:
at least one detent positioned along said at least two guide tracks, said at least one detent being configured to receive said translating member such that when said translating member is received by said at least one detent at least a portion of said plurality of retractor blades are fixed in a corresponding position.

19. The minimally traumatic access port of claim 17, wherein said minimally traumatic access port has at least a first retractor blade and a second retractor blade, wherein said first retractor blade and said second retractor blade have a proximal end and a distal end, said proximal end of said first retractor blade and said proximal end of said second retractor blade being pivotally connected a fastener; said first retractor blade having a first guide track and said second retractor blade having a second guide track, said first guide track aligning with said second guide track such that said at least one translating member passes through said first guide track and said second guide track.

20. The minimally traumatic access port of claim 19, wherein said plurality of retractor blades are configured to be extended or retracted by exerting force on said translating member.
